(19) United States
(12) Patent Application Publication
(10) Pub. No.: US 2018/0306691 A1
(43) Pub. Date: Oct. 25, 2018

(54) Systems and Methods for Visualizing the Interior Morphology and Material Properties of Samples from Surface Deformations

(71) Applicant: The Texas A&M University System, College Station, TX (US)

(72) Inventors: Sevan Goenezen, College Station, TX (US); Yei Mei, College Station, TX (US)

(21) Appl. No.: 15/771,727

(22) PCT Filed: Nov. 18, 2016
(86) PCT No.: PCT/US2016/062877

§ 371 Date: Apr. 27, 2018

Related U.S. Application Data
(60) Provisional application No. 62/257,311, filed on Nov. 19, 2015.

(51) Int. Cl. G01N 3/32; A61B 5/00

(52) U.S. Cl. CPC G01N 3/32; A61B 5/0053; G01N 2203/0089; G01N 2203/0025; G01N 2203/0075; A61B 5/0004

(57) ABSTRACT

Systems and methods disclosed herein enable the characterization of material property distribution across a sample to ensure sample consistency and/or to detect and characterize anomalies including tumors, inclusions, cracks, changes in structural stiffness in samples capable to experience deformation. The boundary displacement data is collected using a plurality of induced deformations at a plurality of angles and locations relative to surfaces/boundaries of the sample, and the data is transformed to determine material properties of the sample using numerical techniques and material modeling.

### CROSS-REFERENCE TO RELATED APPLICATIONS

This application is a 35 U.S.C. § 371 national stage application of PCT/US2016/062877 filed Nov. 18, 2016, and entitled “Systems and Methods for Visualizing the Interior Morphology and Material Properties of Samples from Surface Deformations,” which claims benefit of U.S. provisional patent application Ser. No. 62/257,311 filed Nov. 19, 2015, and entitled “Systems and Methods for Visualizing the Interior Morphology and Material Properties of Samples from Surface Deformations,” each of which is hereby incorporated herein by reference in their entirety for all purposes.

### STATEMENT REGARDING FEDERALLY SPONSORED RESEARCH OR DEVELOPMENT

Not applicable.

### BACKGROUND

Some materials have material properties that vary across and/or through a given sample of the material. This may be especially true in the case of human and/or animal tissue. In order to analyze these materials, magnetic resonance imaging, ultrasound techniques, optical coherence tomography, or computed tomography scans are often employed. These methods may enable imaging of the interior of solids while the solids are actively or passively deformed. Furthermore, these imaging modalities are non-invasive, thus the material properties of a non-homogeneous tissue can be determined non-invasively and in-vivo.

### BRIEF SUMMARY OF THE DISCLOSURE

In an embodiment, a method for determining one or more properties of a material, the method comprising: (A) applying a plurality of forces to a material comprising a plurality of surfaces, wherein the plurality of forces are applied to at least two of the plurality of surfaces; (B) collecting a plurality of data from the sample in response to the application of the plurality of forces in (A); (C) repeating (A) and (B) for a first number of cycles; (D) determining a plurality of values associated with a material property of the sample, wherein the material property is measured across at least a portion of the sample.

In an alternate embodiment, a method of determining material properties comprising: (A) applying a plurality of forces to a material comprising a plurality of surfaces, wherein the plurality of forces are applied to at least two surfaces of the plurality of surfaces; (B) collecting a plurality of data from the sample in response to the application of the plurality of forces in (A); (C) repeating (A) and (B) for a predetermined number of cycles; (D) determining a material property distribution for at least one property across and through the sample, wherein the at least one property comprises a shear modulus, Poisson's ratio, a bulk modulus, a first Lame parameter, or an elastic modulus; (E) comparing the distribution from (D) to an expected value or range of values; (F) determining, based on the comparison at (E), if the sample comprises at least one defect; (G) repeating (A) and (B) on at least a portion of the sample in response to a determination at (F) that the sample comprises at least one defect; and (H) determining, based on (G), at least one of a location, a physical property, and a material property of the at least one defect.

In an alternate embodiment, a method of determining material properties comprising: (A) applying a first plurality of forces to a first number of locations on a material comprising a plurality of surfaces, wherein the first plurality of forces are applied to at least two surfaces of the plurality of surfaces, wherein each force of the first plurality of forces comprises a type, a direction, and an angle relative to a plane; (B) collecting a first plurality of data from the sample in response to the application of the plurality of forces in (A); (C) repeating (A) and (B) for a predetermined number of cycles; (D) applying a second plurality of forces to a second number of locations on a material comprising a plurality of surfaces, wherein the second plurality of forces are applied to at least two surfaces of the plurality of surfaces, wherein each force of the second plurality of forces comprises a type, a direction, and an angle relative to the plane; (E) collecting a second plurality of data from the sample in response to the application of the plurality of forces in (D); (F) determining, based on (B) and (E) a material property distribution for at least one property across and through the sample, wherein the at least one property comprises a shear modulus or an elastic modulus.

### BRIEF DESCRIPTION OF THE DRAWINGS

For a detailed description of the preferred embodiments of the invention, reference will now be made to the accompanying drawings in which:

FIG. 1 is an illustration of an analysis of a uniform mesh according to certain embodiments disclosed herein.

FIGS. 2A-2C illustrate the problem domain for a target shear modulus distribution with boundary conditions applied from the top (FIG. 2A), left (FIG. 2B), and right (FIG. 2C).

FIG. 3A defines the target modulus distribution (based on FIGS. 2A-2C) and is provided for comparison to FIG. 3B-3D.

FIGS. 3B-3D illustrate reconstructed modulus distributions in the absence of noise according to certain embodiments disclosed herein.

FIG. 4 is a graph illustrating the shear modulus values over the horizontal line passing through the center of the inclusion for the target and reconstructed shear modulus given in FIGS. 3A-3D.

FIG. 5A illustrates the target shear modulus distribution and is provided for comparison to FIGS. 5B-5D.

FIGS. 5B-5D illustrates the shear modulus reconstructions in the presence of about 1% noise.

FIG. 6 is a graph illustrating the shear modulus values over the horizontal line passing through the center of the inclusion corresponding to FIGS. 5A-5D

FIG. 7A illustrates the target shear modulus distribution and is provided for comparison to FIGS. 7B-7D.

FIGS. 7B-7D illustrates the shear modulus reconstructions in the presence of about 2.5% noise.

FIG. 8 is a graph illustrating the shear modulus values over the horizontal line passing through the center of the inclusion corresponding to FIGS. 7A-7D.

FIGS. 9A and 9B illustrate modulus distributions where limited surface displacement data was used.

FIG. 10 illustrates the problem domain for a target shear modulus distribution with boundary conditions applied simultaneously from the left and right side.

FIG. 11 illustrates the reconstruction of the shear modulus with 7 paired forces (7 surface displacements on the top boundary edge) and a simulated noise level of 0.1%.

FIG. 12 illustrates the reconstruction of the shear modulus with 7 paired forces (7 surface displacements on the top boundary edge) and a simulated noise level of 1%.

FIG. 13 illustrates a half circle with target shear modulus distribution.

FIG. 14 illustrates the reconstruction of the shear modulus from 5 surface displacements with a noise level of 0.1%.

FIG. 15 illustrates an exemplary system capable of executing according to certain embodiments of the present disclosure.

FIG. 16 illustrates an exemplary method of determining material properties and detecting and characterizing defects according to certain embodiments of the present disclosure.

### DETAILED DESCRIPTION OF THE PREFERRED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices, components, and connections.

### INTRODUCTION

The ability to determine a material parameter distribution in a solid such as human and animal tissue offers the potential to enable the diagnosis and/or evaluation of potentially compromised tissues, and the ability to determine a material parameter distribution in a solid such as materials used in building, construction, aerospace, medical device, and other fields, offers the potential to enable enhanced failure analysis/predictive determination (e.g., crack or defect detection). Embodiments the systems and methods disclosed herein can be used to determine the material properties of a sample of stiff, semi-stiff, or soft material, natural or man-made, and such material properties can be stored for further use and analysis. If there are expected values for the material, such as shear modulus, density, elastic modulus, Poisson's ratio, a bulk modulus, a first Lame parameter, or other viscoelastic, nonlinear hyperelastic, and plastic (plasticity-related) properties, such material properties, as well as physical properties (e.g., location along multiple axes, size, or shape), can be expressed in the form of ranges, targets, and or gradients. Such expected values can be compared to the properties determined in accordance with embodiments described herein to determine if further analysis is needed and/or if the sample complies with the expected results/property ranges.

In an embodiment, the systems and methods disclosed herein are employed to discover material defects by way of property analysis. In other words, analysis using the embodiments of systems and methods disclosed herein can be used to detect both natural and induced (manufacturing/processing) defects such as voids, inclusions, impurities, contaminants, and other defects that may occur within a material and may therefore not be visible (i.e., visible with a naked eye because the defect is contained within the material) on the surface. These defects can be characterized and/or identified by their material properties such as shear or elastic modulus, density, or other material properties, as well as by physical properties such as the shape, size, and position. The analysis process can comprise an iterative process in which a plurality of forces are applied to at least one surface of a three-dimensional sample. Depending upon the result of that initial iteration, more forces, less forces, more surfaces, less surfaces, or different areas of the previously tested surfaces can be applied to the sample to further determine if defects are present and where the defects are located. Using the identification of the presence and location of the defects, the defects can be removed from a sample and/or a manufacturing, harvesting, or inspection process can be adjusted, for example, to add additional inspection steps, features, frequencies, or tests.

Inverse problems in elasticity generally aim to solve for the non-homogeneous material property distribution, which requires knowledge of displacement fields and boundary conditions, (e.g., Neumann and Dirichlet boundary conditions). Material properties, such as the Young's modulus, shear modulus, or Poisson's ratio can be determined for a linear elastic material non-destructively. Other constitutive models, such as viscoelastic, poro-elastic, and hyperelastic models, may comprise additional material parameters. These additional material parameters can potentially be determined from dynamic displacement and large displacement data. The mechanisms employed herein to create surface displacement and identify material properties of an internal area of a sample may be referred to as “forces,” which may be implied as indentations, palpitations, torsion, bending, tension, compression, or combinations thereof. This displacement data can be obtained using embodiments of systems and methods disclosed herein by using palpitations, indentations, bending, torsion, etc. to generate displacement fields and collecting the displacement data on the surface resulting from those experiments and transforming that data using the methods disclosed herein to derive values from which to determine material properties and further characterize materials.

In an embodiment, the forces applied to a sample comprise surface displacements created by repeated application of force in predetermined direction(s) and location(s) with respect to various planes and surfaces of a three-dimensional sample. These forces, or rather the response of the sample to these forces, are used to test the inverse strategy on a problem domain. In one embodiment disclosed herein, this problem domain had one or two stiff circular inclusions in a softer homogeneous background. In other embodiments, defects in a sample that may be less stiff than the background sample can also be detected and characterized. The inverse reconstructions presented here were performed in two dimensional space, and are extended to three-dimensional space. It is observed that the shear modulus reconstruction as well as the detection shape of the circular inclusion improves with an increasing number of surface displacement data sets—that is, with an increasing number of iterations of the application of force on multiple surfaces in multiple directions. Furthermore, with increasing noise level in the surface displacements, the contrast of the reconstructions decreases. Thus it is desirable to keep the noise level in the measured data as low as possible. Using embodiments of the systems and methods disclosed herein, inclusions in various types of soft and stiff materials may be found without making any previous or preliminary assumptions about the location of stiff inclusions, shape or shear modulus values, which opens up the possibility to use this technology for material property distributions other than inclusions in soft backgrounds.

The ability to, using the systems and methods disclosed herein, determine the material parameter distribution of an interior of a solid has potential applicability in a broad range of disciplines. For example, in human or animal tissue mechanics, the change in material properties across a sample or between samples could be correlated to distinguish tissue types, thus could help to classify tissues non-invasively and to monitor their change periodically in the living organism. In alternate embodiments, the change in material properties may indicate an inclusion, which may be a tumor or another growth that may be identified, located, and characterized. Quantifying the material properties of diseased tissues can provide clues on the type and stage of the disease. This can be done as the tissue composition changes with disease development, which is manifested in the macroscopic mechanical response. Another potential application area is in detecting structural failure by monitoring changes in their local mechanical response. Also, in engineered materials, including but not limited to tissues such as cardiovascular grafts among many other engineered tissues, the material properties can be analyzed and the spatial quality/reliability of the manufactured grafts correlated with their material properties, the manufacturing process, the manufacturing line, etc.

As disclosed herein, the solution of the inverse problem employs measured displacement fields, which in general are acquired in the entire interior of the solid. This may conventionally be done using state of the art magnetic resonance imaging, ultrasound techniques, optical coherence tomography, or computed tomography scans. The systems and methods disclosed herein may enable the determination of material properties of solids while the solids are actively or passively deformed. Furthermore, these imaging modalities are non-invasive, thus the material properties of a homogenous or non-homogeneous tissue can be determined non-invasively and in-vivo.

In contrast to embodiments disclosed herein, previous ways to solve the inverse problem in elasticity is by using observations from the exterior of the sample. These observations may be used in the absence of analysis/observations from the interior of the sample. This procedure provides little information to infer the subsurface material property distribution of the sample, and some approaches utilized measured static surface deformations, time harmonic surface deformations using high-speed cameras, and force information. The approaches discussed above utilize a priori information that is generally not available. A priori information may comprise assumptions such as knowing that there even is a defect/object, the location of the object in the solid domain, its size, or shape, and this information may limit the practical application of previous works. Embodiments of the systems and methods disclosed herein are able to characterize materials, with or without defects, and to find and characterize defects when present, in the absence of a priori information.

Presented herein are embodiments of systems and methods to the inverse problem in finite elasticity for the non-homogeneous shear modulus distribution solely from measured surface deformation fields. The inverse problem is posed as a constrained optimization problem under regularization and solved utilizing the adjoining equations. It is appreciated that the equations disclosed herein are example equations and that variations may also be possible depending upon the application.

In embodiments disclosed herein, a plurality of simulated “measured” surface displacement fields are created by inducing temporary indentations on the exterior of the specimen, e.g., the sample is not damaged by the application of these forces. In one example, surface displacement fields are induced by the application of force can be determined using a plurality of digital cameras (more than 2 cameras for 3D view). The surface displacement data is employed to determine the heterogeneous material property distribution by solving the inverse problem without making any assumptions about the composition of materials, i.e., variations of material properties in the problem domain. This approach may not only determine material properties of a sample that is homogenous or (intentionally) non-homogenous in its properties, but may also determine the location size, dimensions, and shape and to determine quantitative values for the material properties of defects that represent tumors, including those that may develop in breast tissue, or manufacturing defects that may also include voids or contaminants.

Embodiments described herein comprise a combination of features and advantages intended to address various shortcomings associated with certain prior devices, systems, and methods for determining material properties of an interior of a sample, including detecting irregularities and characterizing those potential defects. The foregoing has outlined rather broadly the features and technical advantages of the invention in order that the detailed description of the invention that follows may be better understood. The various characteristics described above, as well as other features, will be readily apparent to those skilled in the art upon reading the following detailed description, and by referring to the accompanying drawings. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims

Using the systems and methods disclosed herein, the inverse problem in elasticity is addressed for the shear modulus distribution using non-destructive information. This methodology does not require any a-priori information about the problem domain. However, if a-priori knowledge is available, it may be included in the analysis. The techniques here are based on optimization methods and finite element techniques, and the shear modulus distribution is represented as unknowns on the mesh nodes and interpolated with finite element shape functions. In an embodiment, the number of unknown shear modulus values are equal to the total number of finite element nodes. This method was evaluated in one example on a problem domain comprising one and two inclusions embedded in a homogeneous background, and the shear modulus distribution was recovered using simulated surface displacements. Additionally, noise was added to the data to mimic noise from measured surface displacements from recorded digital camera images.

Methods

Inverse Problem Formulation Utilizing Measured Surface Displacement

The solution of the inverse problem is highly unstable due in part to noisy displacement data, and thus is formulated as a constrained optimization problem. More precisely, the misfit between measured and computed surface displacements are minimized under the “control” of a regularization term. One “natural” way to formulate the inverse problem statement is as follows. Find the shear modulus distribution μ such that the objective function:

$$F = {\sum }_{i = 1}^{n} {\int }_{{Γ}_{i}} {( {u}^{i} - {u}_{meas}^{i} )}^{2} d Γ + α Reg ( μ ) \qquad ( 0.1 )$$

is minimized under the constraint of the forward elasticity problem. The first term is the displacement correlation term, minimizing the square of the misfit between the computed uⁱ and measured uₘₑₐₛⁱ surface displacements on the problem boundary. The summation indicates that this formulation can accommodate multiple surface displacements, where n denotes the total number of observations. It is emphasized that the boundary integral Γᵢ is intentionally augmented with the index i to accommodate surface displacement data on varying boundaries. This is because it may not be feasible or practical to observe data on the same sub-boundary domain for each experiment. The second term is the so called regularization term to penalize oscillations in the final solution of the shear modulus distribution. The particular regularization type is disclosed herein.

The inverse problem formulation in Equation (1.1) differs from previously seen formulas in that the predicted and measured displacements are correlated on the surface while the referenced approaches correlate the displacements in the entire interior of the problem domain. Discretizing Equation (1.1) with finite element techniques is straight forward. This will be demonstrated for one displacement field to reduce notations, this is

$$F = {\int }_{Γ} {( Δ u )}^{2} d Γ + α Reg ( μ ) \qquad ( 0.2 )$$

where Δu=u−uₘₑₐₛ. The finite element interpolation yields

$$F = {\sum }_{e = 1}^{N} {\int }_{{Γ}_{e}} {[ {\sum }_{j = 1}^{{N}_{e}} Δ {u}_{j}^{e} {N}_{j}^{e} ( x ) ]}^{2} d Γ + α Reg ( {\sum }_{k = 1}^{{N}_{n}} {μ}_{j} {N}_{j} ( x ) ) \qquad ( 0.3 )$$

where N, Nₑ, and Nₙ denote the total number of finite elements on the boundary, the number of nodes on each element, and the total number of mesh nodes in the problem domain, respectively. Further, Nⱼ^{e}(x) denotes the shape function for the j-th node corresponding to the e-th linear triangular element. While this approach appears to be reasonable, an alternative formulation was employed to facilitate implementation. In particular, domain integrals were used over finite elements at the boundary, given by

$$F = {\sum }_{e = 1}^{\overline{N}} {\int }_{{Ω}_{e}} {[ {\sum }_{j = 1}^{{\overline{N}}_{e}} Δ {u}_{j}^{e} {N}_{j}^{e} ( x ) ]}^{2} d Ω + α Reg ( {\sum }_{k = 1}^{{N}_{n}} {μ}_{j} {N}_{j} ( x ) ) \qquad ( 0.4 )$$

where N̅ denotes the total number of domain elements at the boundary and N̅ₑ̅ denotes the number of element nodes on the boundary of the corresponding elements. It is noted that only displacement information on the boundaries are assumed to be known, despite the integration over element domains. This “unnatural” approach has been executed according to the embodiments disclosed herein to use the inverse solver written for minimizing the misfit in displacements over the volume integral. Thus, one is able to infer and determine the interior material property distribution from a rich surface displacement data set according to certain embodiments disclosed herein.

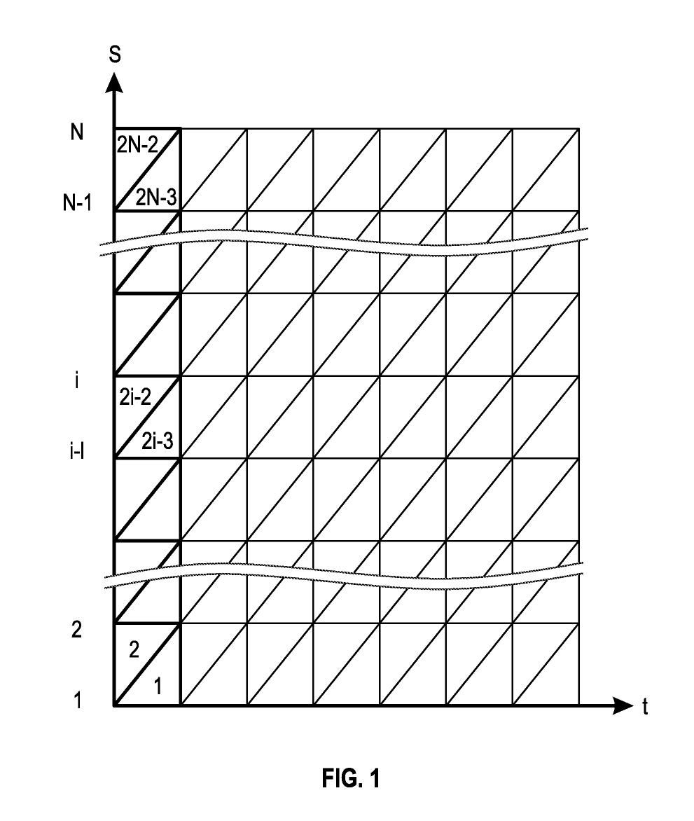

FIG. 1 is an illustration of an analysis of a uniform mesh that can be utilized to discretize the problem domain to numerically solve physics-based mathematical equations. In particular, FIG. 1 illustrates the implications of using Equation (1.3) versus Equation (1.4) on a uniform mesh. The coordinate “s” spans along one boundary edge of a problem domain in two dimensional space and the other boundary edges were discarded to simplify the analysis, the boundary edge is indicated by the bold elements 102 in FIG. 1. The width of the boundary elements along the “t” coordinate is denoted by “a,” and the height along the “s” coordinate of the elements is denoted by “b.” Evaluating Equation (1.4) for the boundary elements given in FIG. 1 yields:

$$F = \frac{ab}{6} Δ {u}_{1}^{2} + {\sum }_{i = 2}^{N - 1} ( \frac{ab}{4} Δ {u}_{i}^{2} + \frac{ab}{12} Δ {u}_{i - 1} Δ {u}_{i} ) + \frac{ab}{12} Δ {u}_{N}^{2} + α Reg ( {\sum }_{k = 1}^{{N}_{n}} {μ}_{j} {N}_{j} ( x ) ) \qquad ( 0.5 )$$

and evaluating Equation (1.3) for the boundary integral yields:

$$F = \frac{b}{3} Δ {u}_{1}^{2} + {\sum }_{i = 2}^{N - 1} ( \frac{2 b}{3} Δ {u}_{i}^{2} + \frac{b}{3} Δ {u}_{i - 1} Δ {u}_{i} ) + \frac{b}{3} Δ {u}_{N}^{2} + α Reg ( {\sum }_{k = 1}^{{N}_{n}} {μ}_{j} {N}_{j} ( x ) ) \qquad ( 0.6 )$$

As such, it is determined that the displacement correlation term in Equation (1.5) contains the element width a and element height b in every term, thus can be factorized. Further, dividing Equation (1.5) by a factor of ab will not change the final solution, i.e. the location of the minimum does not change. Thus Equation (1.5) may be re-written as

$$F = \frac{1}{6} Δ {u}_{1}^{2} + {\sum }_{i = 2}^{N - 1} ( \frac{1}{4} Δ {u}_{i}^{2} + \frac{1}{12} Δ {u}_{i - 1} Δ {u}_{i} ) + \frac{1}{12} Δ {u}_{N}^{2} + \overline{α} Reg ( {\sum }_{k = 1}^{{N}_{n}} {μ}_{j} {N}_{j} ( x ) ) \qquad ( 0.7 )$$

In Equation (1.7), α̅ was substituted for

$$\frac{α}{ab} .$$

Similarly, in Equation (1.6) b can be factorized from the displacement correlation term and divide the entire expression by 4b, resulting in

$$F = \frac{1}{12} Δ {u}_{1}^{2} + {\sum }_{i = 2}^{N - 1} ( \frac{1}{6} Δ {u}_{i}^{2} + \frac{1}{12} Δ {u}_{i - 1} Δ {u}_{i} ) + \frac{1}{12} Δ {u}_{N}^{2} + \overline{α} Reg ( {\sum }_{k = 1}^{{N}_{n}} {μ}_{j} {N}_{j} ( x ) ) \qquad ( 0.8 )$$

where α̅ is substituted for

$$\frac{α}{b} .$$

It was also observed mat, for a uniform mesh, the objective function for the domain integral in Equation (1.7) and the boundary integral in Equation (1.8) differ by their weights. It is noted that these weights are independent of the element size for a uniform mesh.

The particular regularization type used in this work is the total variation diminishing regularization given by

$$Reg ( μ ) = {\int }_{Ω} \sqrt{{\vert ∇ μ \vert}^{2} + {c}^{2}} d Ω \qquad ( 0.9 )$$

where c=0.01 is a small constant to avoid singularities when calculating the gradient of the objective function with respect to the unknown shear modulus. The advantage of total variation diminishing regularization is that it does not smooth out interfaces between object boundaries, e.g. sharp transitions in the shear modulus value between objects are preserved, which allows for the factoring in of the sharp transition in the property analysis. The weight of the regularization term is controlled by the regularization factor α, and is noise-dependent. In particular, the higher the noise level the higher the choice of the regularization factor. It can be determined using the L-curve method, Morozov's discrepancy principle, or based on a smoothness criteria. In some embodiments, the smoothness criterion provides superior reconstructions as compared to the Morozov's discrepancy principle or the L-curve method. It assumes that some small region in the target shear modulus distribution is smooth. Then the optimum regularization factor α takes on the smallest value possible that yields a smooth shear modulus reconstruction in some small sub-region in the problem domain. In general, choosing a regularization factor that is smaller than the optimum will result in shear modulus reconstructions being oscillatory, while choosing a larger regularization factor will overly smooth the shear modulus reconstruction. Other regularization types can be adopted here or alternative approaches to control noise amplification using penalty functions or a covariance kernel.

The inverse problem software is written efficiently in Fortran and utilizes the OpenMP shared memory platform. The user provides an input file that specifies the problem, including mesh information (connectivity, coordinates, element types, etc.), boundary conditions, and measured data, among other input variables. In a pre-processing step, this input file is read by the program, and the parameters are estimated iteratively when the program runs. The matrix/vector systems are solved with the PARDISO solver which utilizes powerful direct solution algorithms for sparse systems.

The BFGS method is employed to solve the constrained optimization problem. The input arguments for the limited BFGS subroutine are the gradient of the objective function with respect to the unknown nodal shear modulus distribution and the functional value at each minimization call. The subroutine then returns with an updated estimate of the parameters and this process is repeated until the change in the objective function is smaller than a specified tolerance. More details on this formulation are given in the following section. The gradient of the objective function is determined efficiently by the adjoint equations. The data generated from inducing displacement fields in samples is employed as disclosed herein and using the formulas herein to determine the material property distribution (, i.e. shear modulus distribution or other elastic constants), which has implications on detecting interior objects and their size and shape.

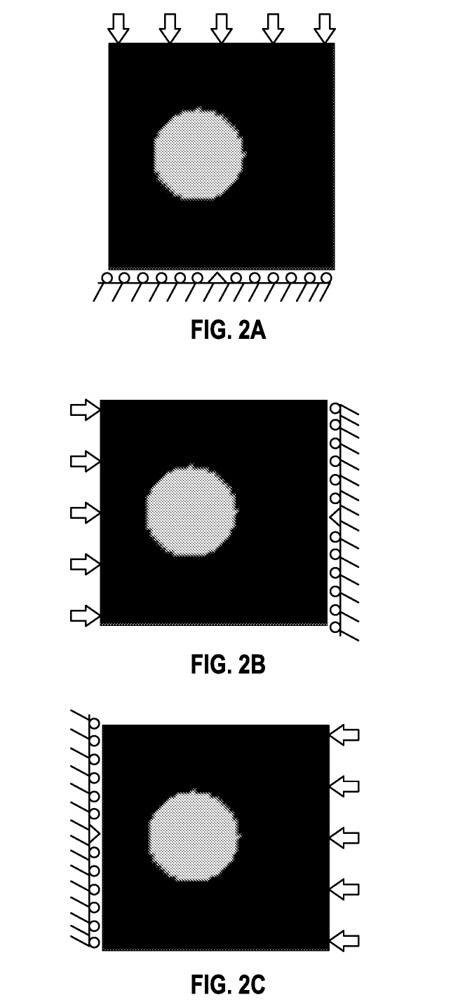

FIGS. 2A-2C show the same target shear modulus distribution with indentations applied on the top (FIG. 2A), left (FIG. 2B), and right (FIG. 2C) edges, respectively. Each arrow represents one simulated experiment inducing one surface displacement response. Surface displacements are referred to displacements on the boundary of the specimen (also referred to as boundary displacements) and visible to the eye. Thus, in this two dimensional target problem domain, displacement data would be visible only on the boundary edges and not throughout the interior domain and inclusion. The indentations in this problem domain are applied as prescribed displacements on a certain finite element node.

In an embodiment, the equation (1.4) is employed to illustrate the feasibility to solve the inverse problem in elasticity, this equation will be tested with simulated displacement data, representing “measured” displacement data on the surface of the problem domain. The simulated displacement data will be created by solving the finite element forward problem for this given problem domain (includes geometry and prescribed shear modulus distribution) and boundary conditions. FIGS. 2A-2C illustrates a problem domain comprising a square domain of unit length on each side. The shear modulus distribution is given with a stiff inclusion 204 having a shear modulus value of μ=5 and a soft homogeneous background 206 having a shear modulus value of μ=1. The inclusion diameter is given with 0.4 units. The arrows on the boundary represent the indentation at various locations to deform the problem domain. From FIGS. 2A-2C, the indentations are applied on the top (2A), left (2B), and right (2C) edges, respectively. The motion of the corresponding opposite side is restricted in the direction of the indentation and the center node on that edge is fixed in all directions to avoid rigid body.

In an embodiment, a bottom boundary 208 is fixed in a vertical direction and can freely move in a horizontal direction, and the center node 210a (indicated by a triangle) of a plurality of nodes 210 (indicated by circles) on that edge is fixed in all directions to avoid rigid body motion. The arrows on the boundary illustrate representative indentations 202 at various locations to deform the problem domain. In other words, each of these indentations 202 results in a deformation field. FIG. 2A is intended as a non-limiting example of the application of deformation fields, the fixed and movable directions depending upon the direction in which the deformation fields are applied, and to which part of the sample the deformation fields are applied. Data is collected along multiple boundary edges (this would relate to the sample's surface for a three dimensional specimen) of a sample depending upon the size, shape, and a composition of the sample. In this example, each indentation induces a displacement of 0.05 units on the corresponding node and perpendicular to the corresponding boundary. It is noted that only 15 arrows are shown in FIGS. 2A-2C for illustration purposes in 2-dimensions, while a total of 27 displacement fields are actually created for this study.

In an embodiment, the material was modeled in two dimensional space, in particular in plane strain for an incompressible material. The finite element mesh consists of 7200 linear triangular elements and the displacement as well as the pressure variables are interpolated with linear shape functions. This approach is neither confined to incompressible materials nor to the plane strain assumption and can be expanded to models beyond. Numerical instability in the pressure variable due to the incompressibility constraint and the violation of the Ladyzhenskaya-Babuska-Brezzi (LBB) conditions has been addressed using stabilized finite element methods.

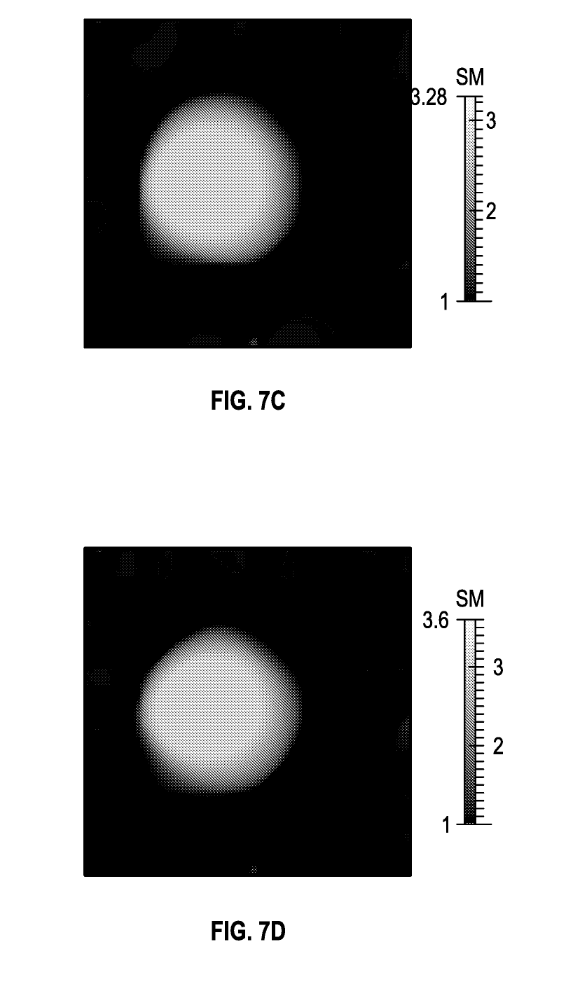

FIG. 10 represents a square domain with 1 cm side lengths and a very small off centered inclusion with a radius of 0.1 cm. The target shear modulus distribution in the background is 10 kPa and in the stiff inclusion 50 kPa. The color bar in FIG. 10 was rescaled by a factor of 10 to account for the right unit representation. The bottom edge was fixed in all directions, and pairwise force indentations of about 0.05 N were applied on both sides, while the surface displacements are measured on the top edge (green line). The pairwise forces are applied horizontally and face opposite to each other when connection them with a horizontal line. Simulations were performed in plane strain for an incompressible material with 7200 linear triangular finite elements and solved using stabilized finite elements as discussed above. This approach is neither confined to incompressible materials nor to the plane strain assumption and can be expanded to models beyond.

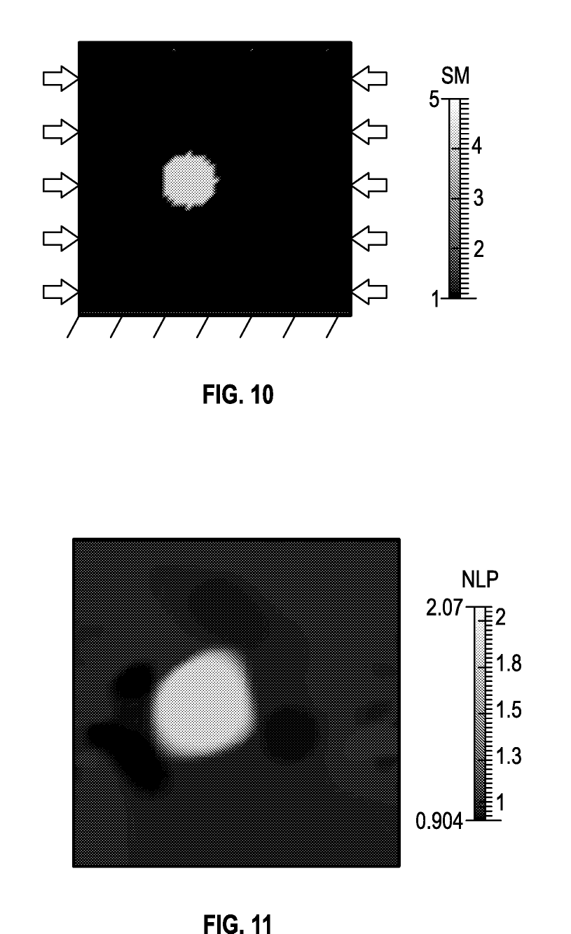

FIG. 13 represents the target shear modulus distribution of a semi-circle to test material property reconstruction using different geometries and with two inclusions. The radius of the semi-circle is 7.5 cm and the radius of each stiff inclusion is 1 cm. The problem domain is discretized with 7632 linear triangular elements and simulations were performed in plane strain for an incompressible material. This approach is neither confined to incompressible materials nor to the plane strain assumption and can be expanded to models beyond. The target shear modulus of the background and inclusions are 5 kPa and 25 kPa, respectively. These values are close to measurements of fatty breast tissue and tumors published in the literature. The bottom edge is fixed and indentations are applied with a small force of about 0.27 N at 5 locations and at varying angles on the top curved edge.

In an embodiment, order to mimic displacement data obtained from digital camera images, 0.1%, 1% and 2.5% white Gaussian noise was added to the surface displacement field obtained from the solution of the forward problem which is significantly higher than the measurement error obtained with the DIC system according to DIC system manufacturers. The noise level is defined by equation (1.10):

$$100 \% \sqrt{\frac{{\sum }_{i = 1}^{\overline{N}} {( {( {u}_{meas}^{surf} )}_{i} - {( {u}_{surf} )}_{i} )}^{2}}{{\sum }_{i = 1}^{\overline{N}} {( {u}_{surf} )}_{i}^{2}}} \qquad ( 1.10 )$$

where (uₘₑₐₛ^{surf})ᵢ and (μ_{surf})ᵢ denote the measured and computed displacement (for given target shear modulus distribution) at node i, respectively.

Results

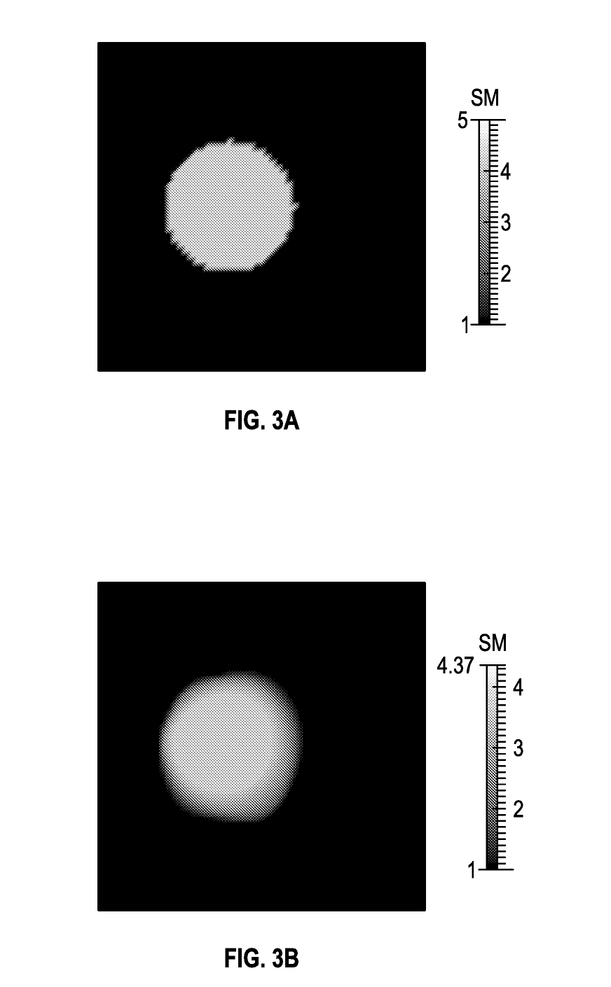

FIG. 3A shows the target shear modulus distribution and FIGS. 3B-3D reconstructions in the absence of noise, FIG. 3B illustrates the reconstructed shear modulus using 9 displacement fields, FIG. 3C illustrates the reconstructed shear modulus using 15 displacement fields, and FIG. 3D illustrates the reconstructed shear modulus using 27 displacement fields.

FIGS. 3B-3D represent the shear modulus reconstructions from 9, 15, and 27 boundary displacements (each representing one simulated experiment) without noise, where the displacements are induced by indentations of 0.05 displacement units. A regularization factor of α=10⁻¹¹ has been selected for all shear modulus reconstructions without noise. The indentations are evenly distributed on the three “visible” sides of the specimen, i.e. for the case with 9, 15, and 27 displacement fields there are 3, 5, and 9 indentations on each edge, respectively (not pictured). The target shear modulus distribution is given in FIG. 3A to facilitate comparison with the shear modulus reconstructions. The shear modulus is well recovered using only 9 surface displacement fields as shown in FIG. 3B. The shear modulus ratio of inclusion to background is about 4.3. With increasing number of displacement fields in FIGS. 3C and 3D as compared to FIG. 3B, the circular shape of the inclusion improves as well as the shear modulus values overall.

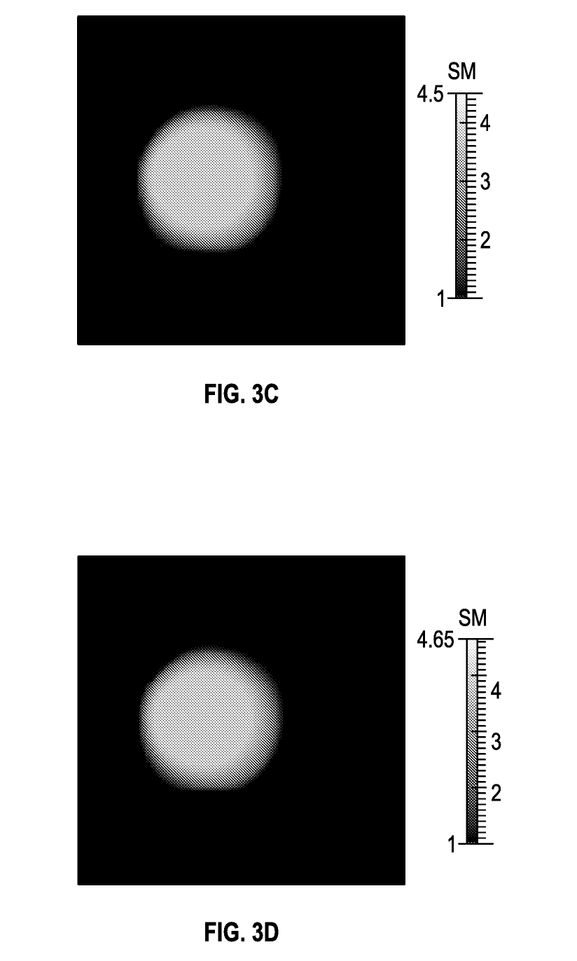

FIG. 4 illustrates a shear modulus plot taken through the center of the inclusion. In FIG. 4, the shear modulus distribution corresponding to FIGS. 3A-3D is plotted along a horizontal line through the center of the inclusion.

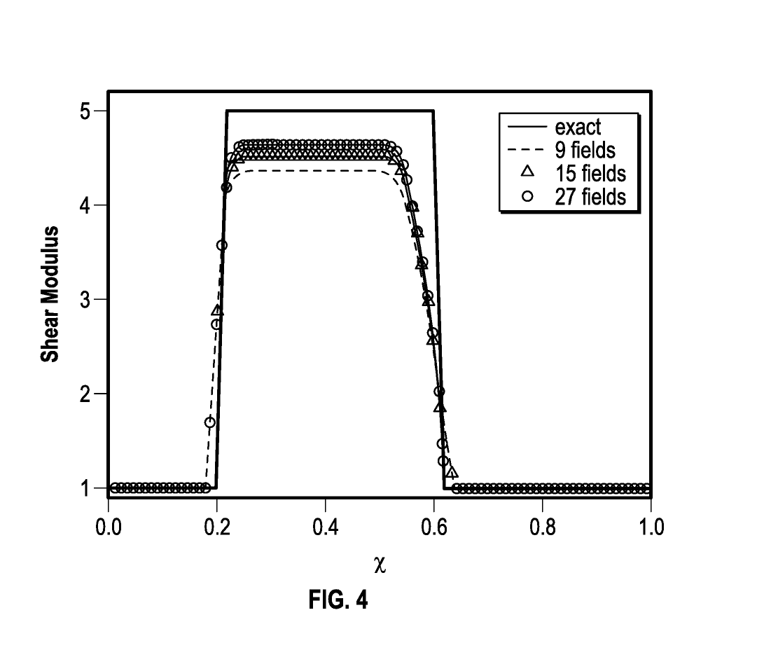

FIGS. 5B-5D illustrate reconstructed shear modulus distributions in the presence of about 1% noise. FIG. 5A illustrates the target shear modulus distribution, FIG. 5B presents the reconstructed shear modulus using 9 displacements, FIG. 5C presents the reconstructed shear modulus using 15 displacement fields, and FIG. 5D illustrates the target shear modulus using 27 displacement fields. FIGS. 5B-5D represent the shear modulus reconstructions from 9, 15, and 27 surface displacement fields, respectively, where each displacement field contains about 1% noise. A regularization factor of α=6·10⁻¹¹ has been selected for all shear modulus reconstructions with 1% noise. The shear modulus ratio of inclusion to background is about 3.54 with 9 surface displacement fields and increases to about 3.8 using 27 surface displacement fields. Furthermore, the shape of the inclusion becomes more circular with an increasing number of surface displacement fields.

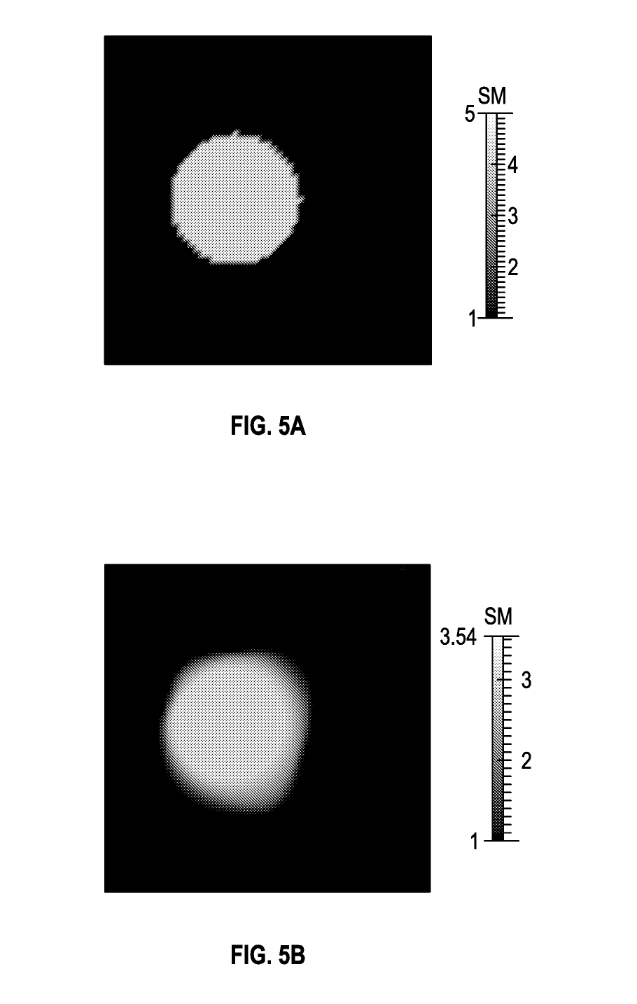

FIG. 6 is a plot of the shear modulus values over the horizontal line passing through the center of the inclusion for the reconstructions given in FIGS. 5B-5D, where the “exact” line indicates the target inclusion in FIG. 5A.

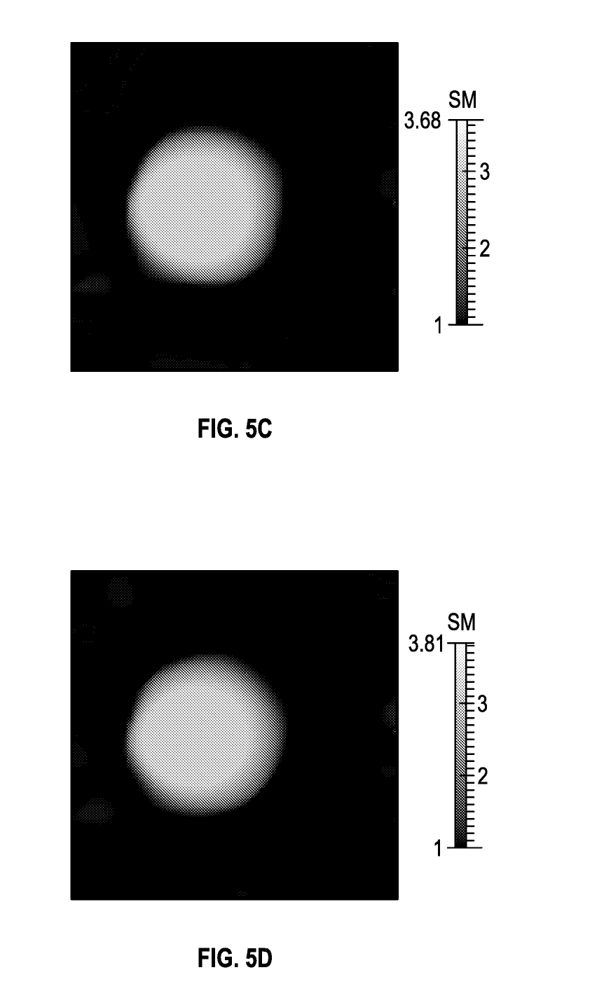

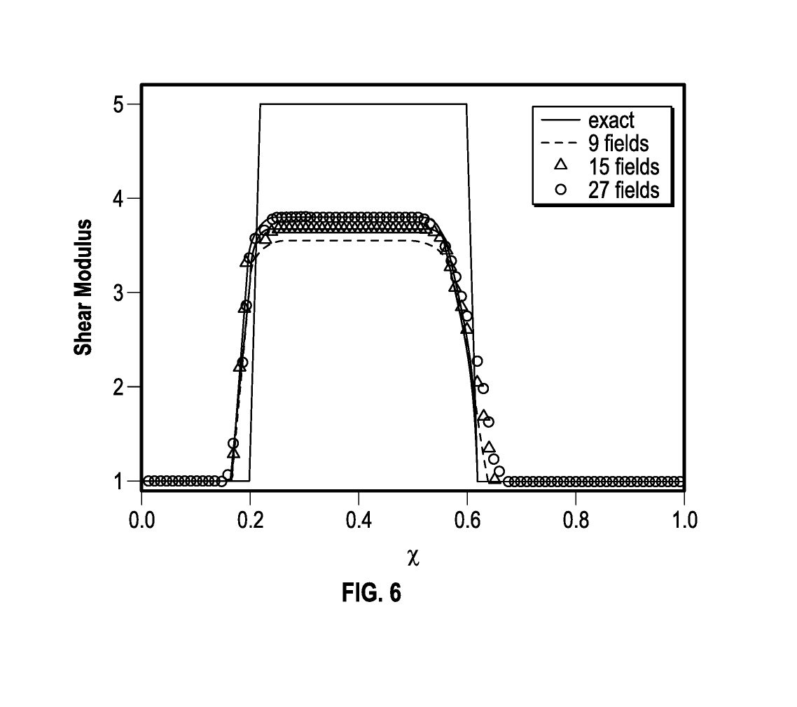

FIGS. 7B-7D show the reconstructed shear modulus distributions in the presence of about 2.5% noise. FIG. 7A presents the target shear modulus distribution, FIG. 7B shows the shear modulus reconstruction using 9 displacements, FIG. 7C shows the shear modulus reconstruction using 15 displacements, and FIG. 7D shows the shear modulus reconstruction using 27 displacement fields. The corresponding shear modulus plot over the horizontal line through the center of the inclusion is given in FIG. 8. A regularization factor of α=10⁻¹⁰ has been selected for all shear modulus reconstructions with about 2.5% noise. The shear modulus ratio of inclusion to background is about 3.25 utilizing 9 surface displacements and increases to 3.6 utilizing 27 surface displacements. The shape of the circular inclusion improves with an increasing number of surface displacements. The shear modulus ratio of inclusion to background does not improve when increasing the total number of surface displacements from 9 to 15 (see FIGS. 7B and 7C respectively), while the shape of the inclusion clearly improves and becomes more circular.

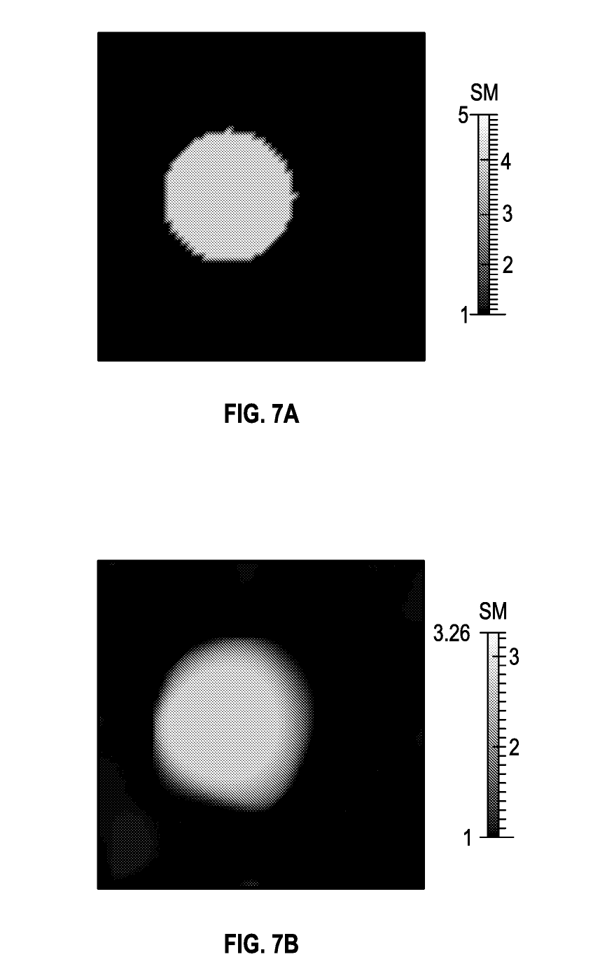

FIGS. 9A and 9B illustrate modulus distributions where limited surface displacement data was used. In practical applications, digital cameras may have only a limited view on the specimen's surface. Thus, the displacement data may only be known in partial boundary regions. Two embodiments are disclosed herein. In the first case, 5 indentations are applied on the top edge, evenly distributed. In the second case, 5 indentations are applied on the top and left edge each. In both cases, the displacement data was used only on the boundary edge where the indentation was applied and 2.5% noise was introduced into the surface displacement field. The reconstructed shear modulus distribution for case one is shown in FIG. 9A and for case two is shown in FIG. 9B for a regularization factor of α=10⁻¹¹. As shown in FIGS. 9A and 9B, it is feasible to recover the shear modulus distribution with limited surface data. However, the shape of the inclusion becomes worse and the shear modulus value decreases. This is because using partial surface deformations will reduce the “richness” of the data set, and could be addressed by including additional surface displacements induced by shear forces and indentations that are not perpendicular to the boundary edge.

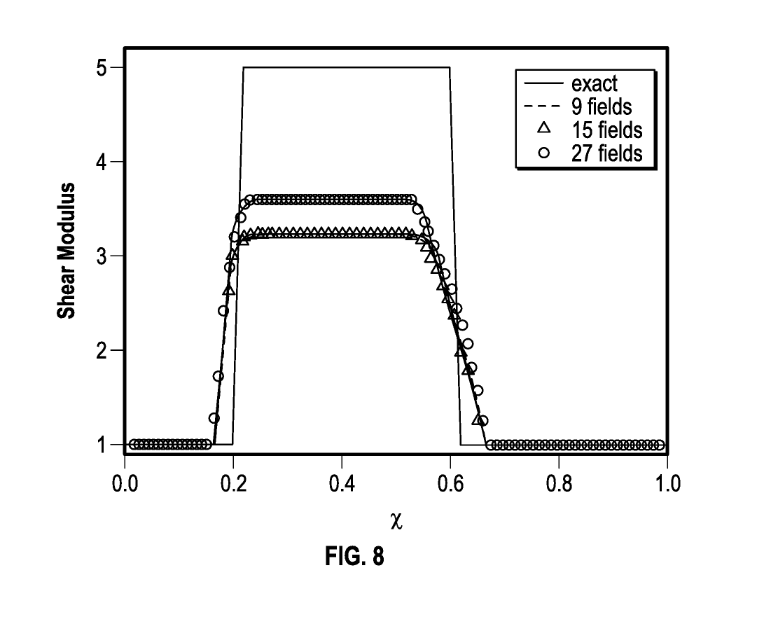

FIG. 11 shows the shear modulus reconstruction corresponding to simulated experiments obtained with the target shear modulus distribution in FIG. 10. A total set of 7 displacements on the top surface/boundary were utilized for the inversion that were induced by applying pair-wise force indentations on the left and right boundary edge. Additionally, to simulate measurement noise, about 0.1% white Gaussian noise was added to the top surface displacement data set. The regularization factor is set to α=10⁻¹¹. As shown in FIG. 11 it is possible to recover the shear modulus distribution from very limited surface displacements in the presence of noise. The color bar must be scaled by a factor of 10 to obtain units of kilo Pascal (kPa). The location of the recovered inclusion is accurate, the shape and values are somewhat off from the actual small inclusions. However, taking into consideration the limited amount of data utilized, the possibility to detect an inclusion of that size is remarkable. Since force indentation was used, the shear modulus reconstructions are absolute as further discussed later on.

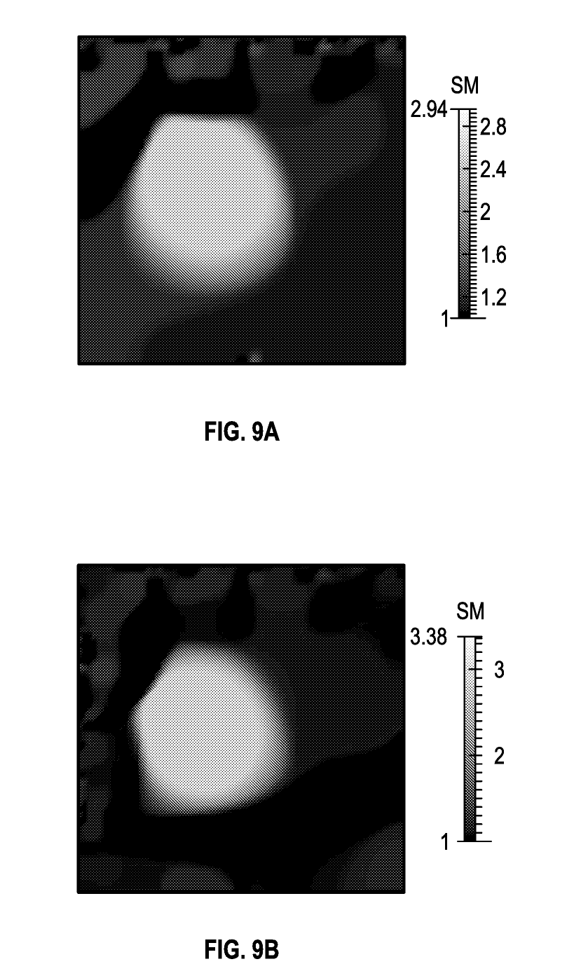

FIG. 12 shows the shear modulus reconstruction corresponding to simulated experiments obtained with the target shear modulus distribution in FIG. 10. A total set of 7 displacements on the top surface/boundary were utilized for the inversion that were induced by applying pair-wise force indentations on the left and right boundary edge. Additionally, to simulate measurement noise, about 1% white Gaussian noise was added to the top surface displacement data set. The regularization factor is set to α=10⁻¹¹. As shown in FIG. 12 it is possible to recover the shear modulus distribution from very limited surface displacements in the presence of higher noise levels of 1%, which is orders higher than expected in experiments according to manufacturers of the digital image correlation system (DIC) at Dantec Dynamics of Holtsville, N.Y. The color bar is scaled by a factor of 10 to obtain units of kilo Pascal (kPa). The location of the recovered inclusion is accurate, the shape and values are somewhat off from the actual small inclusions. However, taking into consideration the limited amount of data utilized, the possibility to detect an inclusion of that size is remarkable. Since force indentation was used, the shear modulus reconstructions are absolute as further discussed later on.

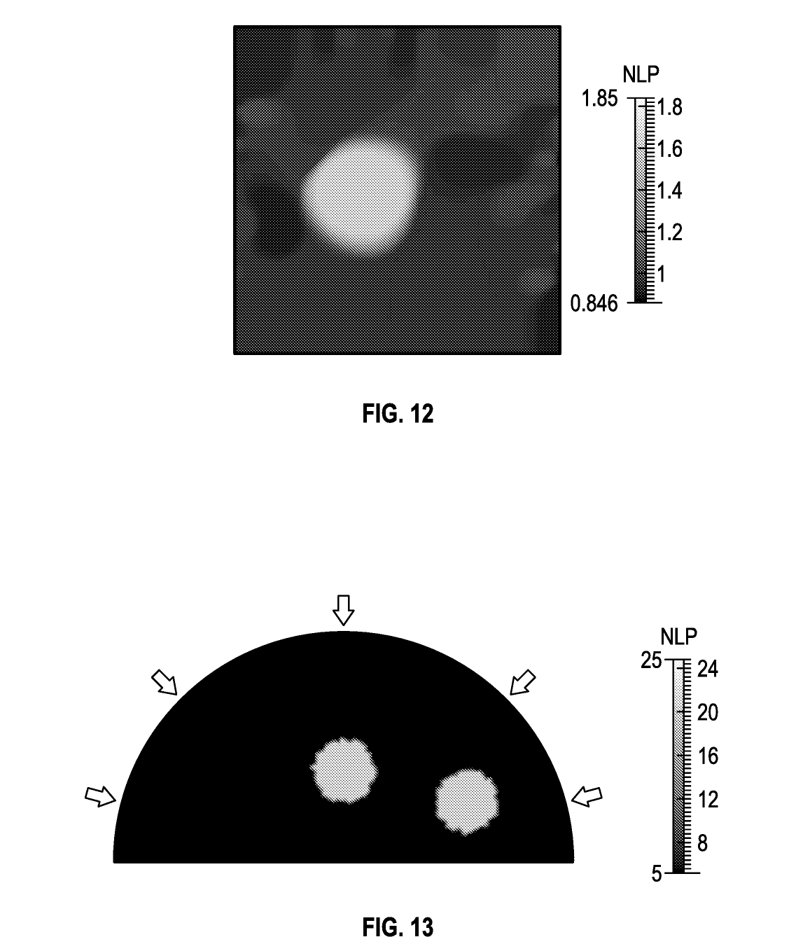

FIG. 14 shows the shear modulus reconstruction corresponding to simulated experiments obtained with the target shear modulus distribution in FIG. 13. A total of 5 force indentations were applied on the curved edge of FIG. 13 to induce 5 surface displacements on the curved edge. To simulate noise, about 0.1% white Gaussian noise was added to the top surface displacement data sets. Since force indentation was used, the shear modulus reconstructions are absolute as further discussed later on. The regularization factor is set to α=10⁻¹¹. As shown in FIG. 14 it is possible to recover the shear modulus distribution for a case where two inclusions are present having distinct stiffness values than their background.

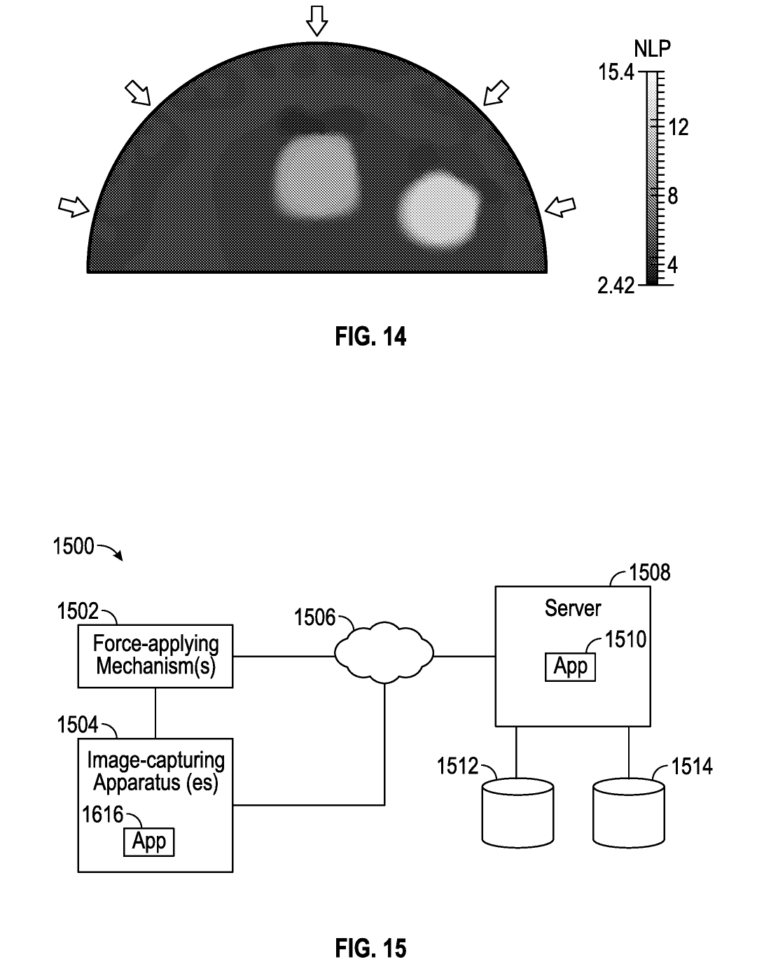

Determination of material properties via solving the inverse problem in elasticity for the shear modulus distribution from displacement data that is measured only on the exterior of a material sample or specimen can be done using a system such as that shown in FIG. 15. More specifically, FIG. 15 illustrates a system 1500 comprising a plurality of force applying mechanisms 1502 and a plurality of image-capturing apparatuses 1504 (e.g., digital cameras), which may include two or more digital cameras positioned at distinct locations relative to different surfaces of a sample. The plurality of digital cameras 1504 record the sample both before and after applying a force, including inducing a deformation field. The images from the digital cameras 1504 are transmitted via a network 1506 to an application 1510 on a server 1508 that is in communication with the digital cameras 1504. The plurality of digital cameras 1504 are configured to capture static positions of the surfaces and/or to capture video to monitor the physical transition(s) of the surface(s) as well as stress/strain, response when the forces are removed, or temperature changes (thermal imaging) induced by the application of force. While the examples here may be discussed as being performed at ambient temperatures, in various embodiments it may be appropriate to perform the method via the system 1500 in a heated or cooled environment.

In some embodiments, the images are processed via software included in the digital cameras 1504 and subsequently processed at that location by an application 1516, and in other embodiments the images are transmitted to the application 1510 via the network 1506 for analysis and processing. In general, the network 1506 can be a wired or wireless network, and the various components can be connected via either wired or wireless connections, including WiFi, Bluetooth, or other known technologies. In an embodiment, the images transmitted from the digital cameras 1504 are processed to infer the three dimensional surface displacements by either of the applications 1516, 1510, and in some embodiments, the application 1516 performs a portion of the analysis and the application 1510 performs a different portion of the analysis in order to determine the material properties across and through a sample, and potentially determine the presence, location, and physical and material properties of the sample. The server 1508 is in communication with one or more data stores 1512, 1514. In one example, a plurality of material properties are stored in the first data store 1512 and the data received by the application 1510 are stored in the second data store 1514.

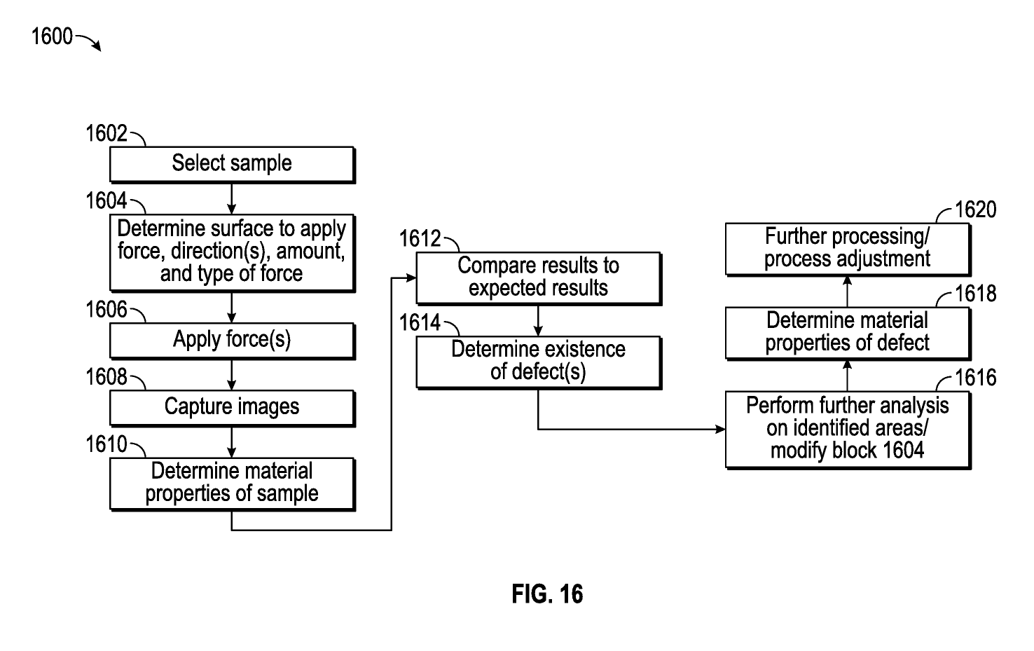

FIG. 16 illustrates a flowchart of a method 1600 according to embodiments disclosed herein. In the method 1600, at block 1602, a sample is selected. This sample can comprise a soft material (e.g., tissue), a rigid material, a semi-rigid material, or combinations thereof (composite) with varying properties. At block 1604, a plurality of determinations that may be related are made, these may comprise a determination of what surfaces of the sample to apply force to, the type of force applied, the direction the force is applied, the amount of force used, and other testing factors as disclosed herein. This information is stored in a data store that is associated with the server 1508 in FIG. 15 or with the force-applying mechanism 1502 or the digital cameras 1504. That is, there may be predetermined ranges of testing or test setups for different types of materials or types of analysis, and this information may be loaded into the system 1500 by referencing the material type or other reference indicator that may pull information from one or more data stores. In some cases, this information is determined at block 1604 based not only on a sample type, but on a shape, size, age, or other factor such as whether the material is naturally-occurring, man-made, or a combination of both.

At block 1606, the plurality of forces are applied as determined at block 1604, and may be applied iteratively in one or more cycles. In some embodiments, the method proceeds back to block 1604 after a predetermined number of cycles (as few as 1) at block 1606, and the parameters determined at block 1604 are recalculated at block 1604. In an embodiment, the predetermined number of cycles may be referred to as a first plurality of cycles, and may be determined based on the material's size, shape, previous data stored as discussed above for the same or similar types and/or shapes of material, or other factors. At block 1608, a plurality of images are captured, and at block 1610, material properties are determined across the sample via transformation and analysis of the images captured at block 1608. The information captured at block 1608 is stored locally or transmitted as discussed above for storage on a remote data store, as may the analysis at block 1610.

In an embodiment, there are expected results for the sample or sample type/dimension/etc. that have been previously stored in a data store. At block 1612, the results obtained at block 1608 via the analysis and transformation at block 1610 is compared by the application to a set of expected results and/or ranges. At block 1614, the application determines whether there are one or more defects present in the sample. In some embodiments, this determination lead to block 1616 where a plurality of forces are applied in a manner similar to that done at block 1606, but different determinations may be made than those made at block 1604. That is, the sample, in its entirety or in part, may have forces applied of a different type, amount, direction, a different number of forces, etc., at block 1616 than at block 1606. In an embodiment, the entire sample is analyzed at block 1606, and a smaller portion of the sample is analyzed at block 1616. In alternate embodiments, the same portion (or all of the sample) is analyzed at both blocks, and in yet other embodiments, a larger portion of the sample may be analyzed at block 1616 than at block 1606. In some embodiments, at block 1618, at least one defect is detected and characterized by its physical and/or material properties, and this information is stored in the data stores discussed above for future reference. Depending upon the results of blocks 1616 and 1618, at block 1620, the sample may have the defect or defects removed or sectioned around for further analysis, and/or a manufacturing or inspection process used to fabricate the sample may be adjusted or have further quality control measures put into place.

In an embodiment, the method 1600 was executed with simulated/modeled surface displacement data, created by solving the equations of equilibrium (forward problem) for a problem domain defined with a stiff inclusion embedded in a softer background using finite element methods. The inclusion was placed off-centered to avoid displacement fields being symmetric in the problem domain, which could be interpreted as some kind of inverse crime. The surface displacements were a direct result of the indentations applied at distinct location on the specimen's boundary. The inverse problem was solved with 5, 9, 15, and 27 surface displacements and added about 0.1%, 1%, and 2.5% noise to the surface displacement data. Depending upon the embodiment, more than 27 displacements (locations where force is applied) may be used.

The following results were obtained using methods disclosed herein: 1) the shear modulus ratio of inclusion to background improves with increasing number of surface displacements, 2) the shape of the reconstructed inclusion improves with an increasing number of surface displacements, 3) the shear modulus ratio of inclusion to background reduces significantly with increasing noise level, and 4) the shape of the reconstructed inclusion deteriorates slightly with increasing noise level. In fact, the shape of the inclusion is well preserved despite of the high noise level which is mainly due to the proper choice of the regularization type. The inclusion size in the target domain had large and small diameter inclusions that were shown to be successfully recovered with the developed techniques. The size of a defect detected using the embodiments of systems and methods disclosed herein as well as the difference in material properties as compared to the background material that is detected may depend upon a variety of factors including, without limitation, the number of cameras employed, the number of forces applied, the number of surfaces the forces are applied to, the amount and/or direction of the forces, the type of forces (torsion, bending, indentations, etc.), as well as the location of the defect(s) in the material as compared to the location(s) of the forces applied (e.g., the surfaces of the material).

The shear modulus reconstructions corresponding to the problem domain given in FIG. 2 are correct up to a multiplicative factor. In other words, multiplying the shear modulus distributions by an arbitrary constant would result in the same displacement field. This is due to the fact that no non-zero Neumann (i.e., traction or force) boundary conditions were utilized in the boundary data. In this example, the indentations were prescribed as displacements. An absolute shear modulus reconstruction can be obtained if the indentation force is known or if the shear modulus value is known somewhere in the problem domain, e.g. through measurements on the surface of the specimen. The shear modulus reconstructions corresponding to the problem domain given in FIG. 10 and FIG. 13 were absolutely (not off by a multiplicative factor) obtained, since the indentations were prescribed as forces. It was also assumed that the indentation displacement was known from simulated experiments.

Alternative techniques to solve the inverse problem in finite elasticity rely mainly on displacement measurements from magnetic resonance imaging, ultrasound techniques, or optical coherence tomography. These techniques can image the interior of the specimen, thus provide displacement data in the entire interior of the specimen. This rich data set reduces the need of utilizing a large number of experiments (displacement data sets). For example, the target shear modulus distribution could be reconstructed utilizing only 1 to 2 displacement fields. This implies that the computational cost reduces significantly as the forward problem and dual problem (i.e., adjoint equations) are solved for each displacement field separately. On the other hand, measuring surface displacements only requires a set of low cost digital cameras to image the surface of the specimen before and after the indentation, together with a digital image correlation (DIC) software or any other system to compute the surface displacements from these images. In one embodiment, a set of digital cameras may comprise two cameras, and in other embodiments, a set may comprise 3 or more cameras. Thus the experimental set up is significantly cheaper as compared to ultrasound or magnetic resonance techniques. Another advantage of using surface displacement data to solve the inverse problem lies in the fact that force indentations can be measured with simple force sensors. This results in absolute reconstructions of the shear modulus distribution and increases in general the information content, thus making the overall solution of the inverse problem “more unique.” Furthermore, this approach results in 3D stiffness maps readily available from the solution of the inverse algorithms in 3D space, as compared to ultrasound or MRI, where 2D images need to be stacked to obtain a 3D map. Finally, ultrasound based stiffness maps are prone to boundary artifacts, while stiffness maps from highly accurate surface displacement fields will not encounter these issues.

The feasibility of solving the inverse problem in finite elasticity for the shear modulus distribution utilizing simulated measured surface displacements was tested, and the shear modulus distribution for a stiff inclusion in a homogeneous background was successfully recovered, assuming that the shear modulus is unknown on the finite element nodes of the problem domain. This method has potential as a novel diagnostic imaging modality to detect tumors surrounded by healthy tissue from their stiffness contrast, to characterize the mechanical material property distribution non-destructively, or to detect interior cracks that are not visible on the specimen's surface. The quality of the shear modulus reconstructions depend on the noise level inherent in measured surface displacement and force data. Furthermore, the reconstruction quality depends on the number of surface displacements (i.e., number of experiments conducted) utilized to solve the inverse problem.

Displacement indentations were prescribed on the first set of simulated experiments corresponding to the problem domain in FIG. 2 and force indentations were applied for simulations corresponding to the problem domain in FIG. 10 and FIG. 13. Applying force boundary conditions allows to reconstruct small inclusions with very limited surface displacement measurements. The displacement at the indentation tip is measured (here from simulated experiments) and is included in the inverse problem solution. The displacement at the indentation can be measured with a micrometer or other devices in experiments.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

1. A method for determining one or more properties of a material, the method comprising:
(A) applying a plurality of forces to a material comprising a plurality of surfaces, wherein the plurality of forces are applied to at least two of the plurality of surfaces;
(B) collecting a plurality of data from the sample in response to the application of the plurality of forces in (A);
(C) repeating (A) and (B) for a first number of cycles;
(D) determining a plurality of values associated with a material property of the sample, wherein the material property is measured across at least a portion of the sample.

2. The method of claim 1, further comprising:
(E) determining, at least one of a location, a size, and a material property of at least two inclusion in the sample based on the determination of the plurality of values in (D).

3. The method of claim 2, further comprising:
(F) determining a material property distribution of the material based on (D) and (E).

4. The method of claim 1, further comprising mapping, based upon (D) and (E) a distribution of at least one material property of the material.

5. The method of claim 1, wherein the predetermined number of cycles comprises a minimum number of cycles needed to determine a plurality of material property measurements across at least a portion of the sample.

6. The method of claim 1, (B) further comprising collecting the plurality of data using a plurality of digital imaging apparatuses in communication with a non-transitory memory, wherein the collected plurality of data is stored in the non-transitory memory during either of steps (B) and (C).

7. The method of claim 3, wherein the communication is established by at least one of a wired connection and a wireless connection

8. The method of claim 1, wherein each force of the plurality of forces is applied at an angle relative to the plane.

9. The method of claim 5, wherein each force of the plurality of forces is applied at a different angle relative to the plane with respect to other applied forces.

10. The method of claim 1, wherein the plurality of forces comprise palpitations, indentations, bending, or torsion.

11. The method of claim 1, further comprising:
(F) determining, based on the determination at (D), a material property across a portion of the sample other than a portion comprising at least one inclusion.

12. A method of determining material properties comprising:
(A) applying a plurality of forces to a material comprising a plurality of surfaces, wherein the plurality of forces are applied to at least two surfaces of the plurality of surfaces;
(B) collecting a plurality of data from the sample in response to the application of the plurality of forces in (A);
(C) repeating (A) and (B) for a predetermined number of cycles;
(D) determining a material property distribution for at least one property across and through the sample, wherein the at least one property comprises a shear modulus, Poisson's ratio, a bulk modulus, a first Lame parameter, or an elastic modulus;
(E) comparing the distribution from (D) to an expected value or range of values;
(F) determining, based on the comparison at (E), if the sample comprises at least one defect;
(G) repeating (A) and (B) on at least a portion of the sample in response to a determination at (F) that the sample comprises at least one defect; and
(H) determining, based on (G), at least one of a location, a physical property, and a material property of the at least one defect.

13. The method of claim 12, wherein the at least one defect comprises an inclusion, a void, a contamination, or combinations thereof.

14. The method of claim 13, wherein the void comprises an internal tear, rupture, or porosity.

15. The method of claim 12, wherein the location comprises a location of the at least one defect relative to at least two surfaces of the plurality of surfaces.

16. The method of claim 12, wherein the physical property comprises a shape, or at least one dimension of the defect.

17. The method of claim 12, wherein the at least one defect is not visible on nor proximate to any surface of the plurality of surfaces.

18. A method of determining material properties comprising:
(A) applying a first plurality of forces to a first number of locations on a material comprising a plurality of surfaces, wherein the first plurality of forces are applied to at least two surfaces of the plurality of surfaces, wherein each force of the first plurality of forces comprises a type, a direction, and an angle relative to a plane;
(B) collecting a first plurality of data from the sample in response to the application of the plurality of forces in (A);
(C) repeating (A) and (B) for a predetermined number of cycles;
(D) applying a second plurality of forces to a second number of locations on a material comprising a plurality of surfaces, wherein the second plurality of forces are applied to at least two surfaces of the plurality of surfaces, wherein each force of the second plurality of forces comprises a type, a direction, and an angle relative to the plane;
(E) collecting a second plurality of data from the sample in response to the application of the plurality of forces in (D);
(F) determining, based on (B) and (E) a material property distribution for at least one property across and through the sample, wherein the at least one property comprises a shear modulus or an elastic modulus.

19. The method of claim 18, wherein the second number of locations is greater than the first number of locations, and wherein at least one of the second plurality of forces comprises a different type, direction, or angle relative to the plane than at least one of the first plurality of forces.

20. The method of claim 18, wherein the second number of locations is less than the first number of locations, and wherein at least one of the second plurality of forces comprises a different type, direction, or angle relative to the plane than at least one of the first plurality of forces.

21. The method of claim 18, wherein the second plurality of forces is applied to a portion of the sample that is smaller than a portion of the sample to which the first plurality of forces are applied.
